(19) United States
(12) Patent Application Publication
(10) Pub. No.: US 2003/0175226 A1
(43) Pub. Date: Sep. 18, 2003

(54) Method for gradual hair lightening

(75) Inventors: Jitendra Patel, Fox River Grove, IL (US); Gerald Patrick Newell, Hoffman Estates, IL (US); Margie Fowler, Elgin, IL (US); Elizabeth Kim, Morton Grove, IL (US); Fe P. Pascual, Elk Grove Village, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/093,569

(22) Filed: Mar. 8, 2002

(51) Int. Cl. A61K 7/135; A61K 7/13

(52) U.S. Cl. 424/62; 8/408

(57) ABSTRACT

A method for lightening the color of hair which comprises subjecting said hair to a number of treatments, having a set time interval between each two consecutive such treatments, wherein each treatment comprises steps a.) and b.) below:

a.) contacting said hair, for a period of about 5 seconds to about 5 minutes with a recently made mixture of:

Part ai: ammonia in gel base or conditioner base at alkaline pH;

Part aii: oxidizing compound in water with conditioning ingredients at acidic pH.

b) rinsing said mixture from said hair with water;

and wherein said number of treatments is between about 2 to about 30; and

wherein said set time interval between each two consecutive treatments is between about 8 hours and 30 days. The hair may also be lightened on a continuous basis by prolonging the intermittent treatment described above. The hair may also be lightened by exposure to the sun during this time period.

### BACKGROUND OF THE INVENTION

The present invention relates to a method for lightening natural hair color while minimizing hair damage.

Many existing hair-lightening products comprise:

1. Concentrated hydrogen peroxide;

2. Ammonium hydroxide; and

3. Potassium persulfate and ammonium persulfate.

The recommended method of application for such products is to first mix all three parts and then apply to dry hair; leave on the hair for 35 to 45 minutes; rinse and dry. This application lightens hair color. However, it damages hair extensively. The damage occurs due to long exposure of the hair to the concentrated peroxide. If the resultant hair coloring effect is not desirable to the consumer, she has to live with it for at least four weeks since other rectifying chemical treatments would damage the hair even more. Depending upon individual hair growth, touch ups may be needed: however, because such touch ups would damage the hair further, they are not recommended.

It is therefore an object of this invention to develop a method of treating hair, so as to lighten hair color while minimizing hair damage.

These and other aspects of this invention will become evident by a detailed description of the invention given below.

Patents and patent applications related to the field of this invention are as follows:

U.S. Pat. No. 4,104,021 which discloses a process in which human hair is dyed in successive treatments at selected intervals with oxidation colors (aromatic primary amines and amino phenols) admixed in each treatment with an oxidizing agent (H₂O₂ or a derivative thereof)—the quantity of oxidation colorant applied in each treatment being substantially the same and the quantity of oxidizing agent being increased from the first to the last treatment to effect a gradual increase in depth of shade—the mixture being allowed to remain on the hair for substantially the same time in each treatment, followed by removal by rinsing.

U.S. Pat. No. 4,529,404 discloses an autoxidizable hair dye preparation capable of coloring or darkening hair when applied thereto and exposed to the atmosphere comprising a mixture of (I) at least one p-phenylene diamine compound, or An acid addition salt thereof, and (II) at least one 1,2,4-benzenetriol compound, each compound optionally containing nuclearly substituted C₁₋₄ alkyl, alkoxy, hydroxyalkyl or halogen. The preparation is preferably applied and exposed to the atmosphere repeatedly until the desired degree of darkening or color build-up is attained.

The preparations of this invention may also contain known additives or assistants such as hair grooming agents, for example quaternized vinyl pyrrolidone copolymers, carboxyvinyl polymers and the like, plasticizers, thickeners, silicone copolymer, foam boosters, preservatives, perfumes and the like.

U.S. Pat. No. 5,968,486 describes a shampoo composition for lightening and highlighting hair which comprises

(i) a peroxygen compound; and

(ii) an anionic sulfonate;

said composition having a pH less than 5. There is also described an invention directed to a method for lightening and highlighting hair which comprises shampooing the hair with a lightening and highlighting effective amount of a composition of the invention.

Co-pending and commonly assigned U.S. patent application Ser. No. 09/558,235, filed Apr. 24, 2000 discloses a hair conditioning composition for conditioning, lightening, and hilighting hair, which comprises

i) peroxygen compound, and

ii) a conditioning agent,

said composition having a pH of 5 or less.

Co-pending and commonly assigned U.S. patent application Ser. No. 09/811,920, filed Mar. 19, 2001 discloses a method for permanently dyeing hair which comprises subjecting said hair to a number of treatments, having a set time interval between each two consecutive such treatments, wherein each treatment comprises steps a.) and b.) below:

a.) contacting said hair, for a period of about 5 seconds to about 5 minutes with a recently made mixture of:

i.) an alkaline composition comprising a dye intermediate in a shampoo base or in a conditioner base; and

ii.) an acidic composition comprising an oxidating compound in a shampoo base or in a conditioner base;

b.) rinsing said mixture from said hair with water;

with the proviso that when a conditioner base is present in a.)i.) above, an independently selected conditioner base is also present in a.)ii.) above; and when a shampoo base is present in a.)i.) above, an independently selected shampoo base is also present in a.)ii.) above;

and wherein said number of treatments is between about 2 to about 30; and wherein said set time interval between each two consecutive treatments is between about 8 hours and 30 days, is described.

### SUMMARY OF THE INVENTION

The invention relates to a method for achieving permanent desired hair color lightening through the daily or frequent use of hair color lightening compositions which comprise a mixture of two compositions, part ai and part aii as described just below:

Part ai: ammonia with gelling agent or conditioning ingredients at alkaline pH;

Part aii: oxidizing compound in water with conditioning ingredients at acidic pH.

### DETAILED DESCRIPTION OF THE INVENTION

Unless otherwise indicated, all percentages used herein are percentages by weight of active material based on the weight of the respective composition.

When used herein % refers to weight % as compared to the total weight percent of the composition that is being discussed. For example, when % is used to discuss the amount of an ingredient that is in part ai, this means weight % as compared to the total weight of part ai. When weight % of the mixture of part ai and part aii is mentioned, this means the weight % as compared to the total weight the mixture of part ai and part aii. When the ratio of part ai to part aii is discussed this means the weight to weight ratio. As used herein the term “recently” means within a very short interval of time such as within a few seconds or minutes, such as within 0.01 seconds to 120 seconds, or within 0.1 seconds to 60 seconds, or within 0.5 second to within 30 seconds or within 2 seconds to within 20 seconds. As used herein “nearly simultaneously” means within about 0.001 second to about 5 seconds, more preferably about 0.01 to about 1 second, more preferably about 0.01 to about 0.5 second. As used herein “physical proximity” means within about 0.01 to about 1 cm, more preferably about 0.1 to about 0.5 cm. Compositions of the invention may be made by means which are known in the art or which are analogous to those which are known in the art. Ingredients which are included in compositions of the invention are known in the art or may be made by means which are known in the art.

The invention relates compositions and to methods for achieving permanent desired hair color lightening through the daily or frequent use of hair color lightening compositions which comprise a mixture of two compositions, part ai and part aii as described just below:

Part ai: ammonia with gelling agent or conditioning ingredients at alkaline pH;

Part aii: oxidizing compound in water with conditioning ingredients at acidic pH.

Part ai comprises from

a) about 0.1 to about 99.9% of an aqueous ammonia gel base or conditioner base;

More preferably, part ai can comprise from

a) about 1 to about 90% of an aqueous ammonia gel base or conditioner base;

Part aii comprises from:

a) about 1 to about 99% of an aqueous base; and

b) about 1 to about 5% of an oxidizing compound.

Part aii more preferably comprises from:

a) about 2 to about 99% of an aqueous base; and

b) about 2 to about 5% of an oxidizing compound.

Aqueous compositions of the present invention can comprise a mixture of part ai and part aii wherein:

Part ai comprises:

a) about 0.1% to about 99.9% of an aqueous ammonia gel base or conditioner base; and

b) a volatile silicone;

Part aii comprises:

a) about 1 to about 99% of an aqueous base;

b) about 1 to about 5% of an oxidizing compound.

The hair treatment compositions of the present invention are hair bleaching compositions which can be use on a frequent or daily basis, for example, in the shower, so as to lighten the color of natural hair.

The hair treatment compositions of the present invention (which are also called bleach compositions herein) have the following advantages:

1. Bleach compositions can be developed without anionic, non-ionic or soap base surfactants.

2. Bleach compositions can be developed with quaternary compounds.

3. Bleach compositions can be developed with fatty alcohols.

4. Bleach composition can be developed with silicones.

5. Bleach compositions minimize the hair damage during hair color lightening.

6. The user can use bleach compositions in the shower.

7. The user has control on the amount of lifting/lightening on the hair using the bleach compositions.

8. The user can use a bleach composition as her daily hair care composition to avoid new out growth of hair.

What follows is a description of the ingredients that can be included in the compositions of the present invention.

Oxidizing Compounds

The oxidizing compound which may be used in the hair color lightening compositions of the present invention may be selected from the group consisting of hydrogen peroxide, urea peroxide, melamine peroxide, sodium perborate, sodium percarbonate and mixtures thereof.

Ammonia

Ammonia may be used in the compositions of the present invention. Such ammonia can come in the form of ammonium hydroxide of varying strengths such as about 10 to about 50% more preferably about 25 to about 30%.

Gelling Agents

Gelling agents, or structurants or thickeners may be employed in part ai of compositions of the invention.

Examples of suitable structurants or thickeners are polymeric thickeners such as carboxyvinyl polymers. A carboxyvinyl polymer is an interpolymer of a monomeric mixture comprising a monomeric olefinically unsaturated carboxylic acid, and from about 0.01% to about 10% by weight of the total monomers of a polyether of a polyhydric alcohol. Carboxyvinyl polymers are substantially insoluble in liquid, volatile organic hydrocarbons and are dimensionally stable on exposure to air. Suitably the molecular weight of the carboxyvinyl polymer is at least 750,000, preferably at least 1,250,000, most preferably at least 3,000,000. Preferred carboxyvinyl polymers are copolymers of acrylic acid cross-linked with polyallylsucrose as described in U.S. Pat. No. 2,798,053. These polymers are provided by B. F. Goodrich Company as, for example, CARBOPOL 934, 940, 941 and 980. Other materials that can also be used as structurants or thickeners include those that can impart a gel-like viscosity to the composition, such as water soluble or colloidally water soluble polymers like cellulose ethers (e.g. methylcellulose, hydroxyethylcellulose, hydroxypropylmethylcellulose and carboxymethylcellulose), guar gum, sodium alginate, gum arabic, xanthan gum, polyvinyl alcohol, polyvinyl pyrrolidone, hydroxypropyl guar gum, starch and starch derivatives, and other thickeners, viscosity modifiers, gelling agents, etc. It is also possible to use inorganic thickeners such as bentonite or laponite clays.

Buffering Agents

The final hair color lightening compositions of the present invention (that is after part ai and part aii have been mixed) have a preferred pH in the range of from about 7.5 to about 12, more preferably from about 8 to about 10.

Buffering agents may be present in part ai and part aii of the present invention.

Further examples of suitable buffering agents are ammonium hydroxide, urea, ethylamine, dipropylamine, triethylamine and alkanediamines such as 1,3-diaminopropane, anhydrous alkaline alkanolamines such as, mono or di-ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-arginine, lysine, oxylysine and histidine and alkanolamines such as dimethylaminoethanol and aminoalkylpropanediol and mixtures thereof. Also suitable for use herein are compounds that form HCO₃⁻ by dissociation in water (hereinafter referred to as ‘ion forming compounds’). Examples of suitable ion forming compounds are Na₂CO₃, NaHCO₃, K₂CO₃, (NH₄)₂CO₃, NH₄HCO₃, CaCO₃ and Ca(HCO₃)₂ and mixtures thereof.

Certain alkaline buffering agents such as ammonium hydroxide and monoethylamine (MEA), urea, and the like can also act as hair swelling agents (HSA's).

Preferred for use as a buffering agent for the color lightening compositions according to the present invention is ammonium hydroxide and/or sodium hydroxide.

In oxidizing kits comprising a portion of peroxide oxidizing agent, which may be present in either solid or liquid form, such as hydrogen peroxide, a buffering agent solution is required to stabilize hydrogen peroxide. Since hydrogen peroxide is stable in the pH range from 2 to 4, it is necessary to use a buffering agent having a pH within this range. Dilute acids are suitable hydrogen peroxide buffering agents. Phosphoric acid is a preferred agent for buffering hydrogen peroxide solutions.

This pH adjustment can also be effected by using well known acidifying agents in the field of treating keratinous fibers, and in particular human hair, such as inorganic and organic acids such as hydrochloric acid, tartaric acid, citric acid, and carboxylic or sulphonic acids such as ascorbic acid, acetic acid, lactic acid, sulphuric acid, formic acid, ammonium sulphate and sodium dihydrogenphosphate/phosphoric acid, disodium hydrogen phosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate/citric acid, sodium tartarate/tartaric acid, sodium lactate/lactic acid, sodium acetate/acetic acid, disodium hydrogenphosphate/citric acid and sodium chloride/glycine/hydrochloric acid and mixtures thereof.

Solvents

Water is the preferred principal diluent or solvent for the compositions according to the present invention. As such, the compositions according to the present invention may include one or more solvents as additional diluent materials. Generally, the solvent is selected to be miscible with water and innocuous to the skin. Solvents suitable for use herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a particularly preferred group includes ethanol, isopropanol, n-propanol, butanol, propylene glycol, ethylene glycol monoethyl ether, and mixtures thereof.

These solvents may be present in part ai and part aii of the invention.

Fatty Alcohols and Thickeners

Fatty alcohols and thickeners may be included in compositions of the invention. Long chain fatty alcohols having from about 11 to about 18 carbon atoms in the long fatty chain can be thickener constituents of compositions of this invention. These alcohols can be used alone, or in admixture with each other. When included in the compositions, the alcohol is preferably present at from about 0.5 to about 10 weight percent of the composition, and more preferably at from about 2 to about 5 weight percent.

Lauryl alcohol, oleyl alcohol, myristyl alcohol, stearyl alcohol, and the like, and mixtures thereof are contemplated herein. In addition, mixtures of natural or synthetic fatty alcohols having fatty chain lengths of from about 11 to about 18 carbons are also useful. Several such mixtures are available commercially, and are exemplified by the material containing a mixture of synthetic alcohols with 12 to 15 carbons in the alkyl chain sold under the trademark NEODOL 25 by Shell Chemical Company, and the material containing a mixture of synthetic alcohols with chain lengths of 12 to 16 carbons sold under the trademark ALFOL 1216 Alcohol by Conoco Chemicals.

Thickening agents suitable for use in the compositions herein may also be selected from the group consisting of oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM), steareth-20 methacrylate copolymer; Aculyn 44 (RTM) polyurethane resin and Acusol 830 (RTM), acrylates copolymer that are available from Rohm and Haas, Philadelphia, Pa., USA. Additional thickening agents suitable for use herein include sodium alginate or gum arabic, or cellulose derivatives, such as methyl cellulose or the sodium salt of carboxymethylcellulose or acrylic polymers.

Fatty alcohols of the above discussed carbon chain lengths which are ethoxylated to contain an average of one or two moles of ethylene oxide per mole of fatty alcohol can be used in place of the fatty alcohols themselves. Examples of such useful ethoxylated fatty acids include ethylene glycol cetyl ether, polyoxyethylene (2) stearyl ether, and the like; the exemplary compounds having CTFA Dictionary names of Ceteth-1 and Steareth-2, respectively.

Volatile Silicones

Volatile silicones may also be employed in the compositions of the invention. The volatile silicone oil is often described as a volatile polyorganosiloxane, and is a liquid material having a measurable vapour pressure at ambient conditions (about 20 to 25° C.). Typically the vapour pressure of volatile silicones lies in the range of from 1 or 10 Pa to 2 kPa at 25° C. Volatile polyorganosiloxanes can be linear or cyclic or mixtures thereof. Preferred cyclic siloxanes include polydimethylsiloxanes and particularly those containing from 3 to 9 silicon atoms and preferably not more than 7 silicon atoms and most preferably from 4 to 6 silicon atoms, otherwise often referred to as cyclomethicones. Preferred linear siloxanes include polydimethylsiloxanes containing from 3 to 9 silicon atoms. The volatile siloxanes normally by themselves exhibit viscosities of below 1×10−5 m2/sec (10 centistokes), and particularly above 1×10−7 m 2/sec (0.1 centistokes), the linear siloxanes normally exhibiting a viscosity of below 5×10−6 m2/sec (5 centistokes). The volatile silicones can also comprise branched linear or cyclic siloxanes such as the aforementioned linear or cyclic siloxanes substituted by one or more pendant —O—Si(CH3)3 groups. Examples of commercially available silicone oils include oils having grade designations 344, 345 244, 245 and 246, (from Dow Corning Corporation) Silicone 7207 and Silicone 7158 (from Union Carbide Corporation) and SF1202 (from General Electric [US]).

The non-volatile oil can comprise non-volatile silicone oils, which include polyalkyl siloxanes, polyalkylaryl siloxanes and polyethersiloxane copolymers. These can suitably be selected from dimethicone and dimethicone copolyols. Commercially available non-volatile silicone oils include Dow Corning 556 and Dow Corning 200 series having a viscosity of at least 50 centistokes.

Non-Volatile Silicones

Non-volatile silicones may be included in compositions of the invention. Non-volatile silicone oils include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile silicones useful in the present compositions are the polydimethyl siloxanes having viscosities from about 10 to about 400 centistokes at 25° C.

Optional Ingredients

The compositions of the present invention can comprise a wide range of optional ingredients. Examples of the functional classes into which these ingredients may fall include: anticaking agents, antioxidants, binders, biological additives, bulking agents, chelating agents, chemical additives, colorants, cosmetic astringents, cosmetic biocides, denaturants, drug astringents, emulsifiers, film formers, fragrance components, humectants, opacifying agents, plasticizers, preservatives, propellants, reducing agents, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include solubilizing agents, sequestrants, and the like.

Other optional ingredients include organic acids. A non-exclusive list of examples of organic acids which can be used as the proton donating agent are adipic acid, tartaric acid, citric acid, maleic acid, malic acid, succinic acid, glycolic acid, glutaric acid, benzoic acid, malonic acid, salicylic acid, gluconic acid, polyacrylic acid, their salts, and mixtures thereof. Non-exclusive lists of examples of mineral acid for use herein are hydrochloric, phosphoric, sulfuric and mixtures thereof.

It has been found by experimentation that hair care products can be used on a daily or frequent basis to achieve durable desired hair color lightening. As noted above, the daily hair care product consists of a mixture of two parts.

Part ai: of an aqueous ammonia in a gel base or conditioning base at alkaline pH

Part aii: an oxidizing compound such as hydrogen peroxide in water at acidic pH.

Part ai is mixed with part aii and applied to hair. To make the product more convenient and fool proof, part ai and aii can be packaged in dual dispensing systems where both parts are mixed outside of the package when dispensed. The mixture is then applied to the wet hair as a hair color lightening composition. Depending upon the amount of lightening desired, the treatment time could be varied from one or two minutes or longer.

Such treatments would add lightening to hair color gradually and without damage due to lower contact time. Each subsequent treatment would add lightening until the desired shade is obtained. Depending upon the concentration of the actives and contact time, a desired shade may be reached in about six to eight treatments. It has also been found that since any one treatment does not exceed the threshold of irreversible damage, the total damage resulting from multiple treatments is lower than the damage from a single conventional treatment. Such a process gives the user control over the amount of color lightening of her hair, and also the option to discontinue further applications if the color lightening delivered is not to her liking.

The following examples, which were made by conventional means, are shown as illustrations only and are not intended to limit the scope of the invention:

Formula #1

| Ammonia gel Part ai | % |
|---|---|
| Stearamidopropyl dimethylamine | 0.50 |
| Dicetyldimonium chloride/PG, 68%/27% | 2.10 |
| Stearyl alcohol and Ceteareth-20, 70% | 1.00 |
| Cetyl alcohol | 3.60 |
| Disodium EDTA | 0.10 |
| Dimethicone 100% | 1.00 |
| DC silicone fluid 245 | 1.80 |
| Kathon CG 1.5% | 0.08 |
| DMDM Hydantoin 55% | 0.10 |
| Fragrance | 0.20 |
| Ammonium hydroxide 28% | 20.00 |
| DI water | to 100.00 |
| PH = 11-12 | |
| Peroxide Conditioner: Part aii | % |
| Liquid Citric acid, 50% | 0.20 |
| Stearamidopropyl dimethylamine | 0.50 |
| Dicetyldimonium chloride/PG, 68%/27% | 2.10 |
| Stearyl alcohol and Ceteareth-20, 70% | 1.00 |
| Cetyl alcohol | 3.80 |
| Disodium EDTA | 0.10 |
| Dimethicone 100% | 1.00 |
| DC silicone fluid 245 | 1.80 |
| Hydrogen Peroxide(35%) | 25.00 |
| DMDM Hydantoin 55% | 0.10 |
| Fragrance | 0.20 |
| Phosphoric acid, 85% | 0.09 |
| DI Water | to 100.00 |
| PH = 3.0 | |

Experiment #1

Take equal amounts of part ai and part aii (Formula #1), mix and apply to the wet or dry hair tresses, allow to remain on the hair tresses for 2 minutes, and rinse well. Measure the change in color delta E using McBeth ColorEye. Repeat the above procedure for subsequent treatments. Collect the hair tresses after 2, 4, 6, 8,10, 12, 14 treatments. Measure the change in color delta E using McBeth ColorEye.

ΔE is difference of color as compared against untreated hair: √(ΔL²+Δa²+Δb²)

| Samples | L value |
|---|---|
| Control (dark brown | 21.66 |
| uncolored Hair) | |
| Clairol bleaching kit - | 44.35 |
| 1 treatment - 30-35 min. | |
| Formula #1 | |
| a. 2 treatments-2 min | 23.78 |
| each | |
| b. 4- total treatments- 2 | 27.12 |
| min each | |
| c. 6 total treatments 2 min | 30.78 |
| each | |
| d. 8 total treatments 2 | 33.25 |
| min. each | |
| e. 10 total treatments 2 | 36.05 |
| min each | |
| f. 12 total treatments 2 | 37.36 |
| min each | |
| g. 14 total treatments 2 | 40.65 |
| min each | |
| h. 16 treatments 2 min | 42.06 |
| each | |

Experiment #2

The following experiment was done to evaluate the extent of hair damage caused by the above treatments. Wet combing evaluation technique was used to assess damage. Better wet combing attribute indicates less damage.

A set of dark brown hair tresses were treated with conventional Clairol bleaching kit and treated with all systems 1-6 Wet combing force was measured by Instron.

| Samples | Wet Combing Force |
|---|---|
| Control (dark brown uncolored | 18 gm force |
| Hair) | |
| Clairol bleaching kit - | 46.35 |
| 1 treatment - 30-35 minutes | |
| Formula#1 | |
| a. 2 treatment-2 min each | 12.24 |
| b. 4-total treatments- 2 min | 11.78 |
| each | |
| c. 6 total treatments 2 min | 12.06 |
| each | |
| d. 8 total treatments 2 min. | 12.56 |
| each | |
| e. 10 total treatments 2 min | 11.89 |
| each | |
| f. 12 total treatments 2 min | 12.69 |
| each | |
| g. 14 total treatments 2 min | 13.35 |
| each | |
| h. 16 total treatments 2 min | 13.89 |
| each | |

These wet combing results indicate that a composition of the invention causes very little damage compared to conventional hair bleaching systems.

Treatment of hair with compositions of the invention as described herein gives said hair good attributes such as lower wet combing force, higher break stress, lower amounts of cysteic acid (which are an indicator of hair damage), good hair color change, less color fading, less damage to hair, and more intense color to hair as described just below. Also described below is a method for applying compositions of the invention at set time intervals so as to avoid root outgrowth, and have less color fading.

Combing Index—

A combing experiment was carried out to evaluate the extent of hair damage caused by various hair treatments. The wet combing evaluation technique was used to correlate hair damage. The combing index was measured to evaluate the extent of hair damage. The higher the index (that is, a number more than one) the lower the hair damage.

Combing Index=Combing force of untreated hair/combing force of hair treated with hair lightening composition.

The above tresses were evaluated via Instron to measure combing force.

| Treatments | Combing Force gm Force |
|---|---|
| Untreated hair | 27.15 |
| Treated with bleach gel | 12.16 |

$$\begin{matrix}Combing lndex = Combing force of untreated hair / \\ combing force of hair treated \\ with hair lightening composition \\ = 27.15 / 12.16 \\ = .23\end{matrix}$$

Wet Combing Force:

After conventional hair color lightening treatment, it is hard to comb the hair. Harder combing indicates more hair damage. The combing force was measured using Instron.

The combing force with new composition of the invention is in the range of 5 to 55 gm force, preferably 5 to 25 and more preferably 5 to 18 gm force.

Break Stress:

After conventional hair color lightening treatment, the hair becomes weak and easy to break. The weak hair is again a sign of damage. The strength of the hair was measured using Instron.

The break stress with a hair lightening composition of the invention is in the range of 0.005 to 0.03 gm force/micron, preferably 0.005 to 0.025 and more preferably 0.005 to 0.018 gm force/micron.

Cysteic Acid

Much of the hair damage associated with conventional hair color lightening treatment comes from the oxidation of cystine residues to the corresponding cysteic acid, with a consequent decrease in the tensile strength of hair as these cross-linkages are destroyed. A good measure of oxidative damage is thus the amount of cysteic acid formed in hair.

Infrared transmission spectroscopy has been used to determine cysteic acid content in hair. The ratio of absorption at 1040 cm−1/absorption at 1240 cm−1 would indicate the extent of damage. Lower the ratio indicates less hair damage.

The ratio of 1040/1240 with new hair lightening compositions of the invention is in the range of 0.01 to 1.5, preferably 0.01 to 1.0, and more preferably 0.01 to 0.5

Hair damage done by hair lightening compositions can be calculated according to the following mathematical formulas:

% Damage=Chemical damage×Physical damage

% Damage=amount of cysteic acid×combing force gm×breaking stress forcegm/micron2×100

$$Input Workspace TL4 \\ Date : 09 / 11 / 2003 Number : 10093569 Folder : 02$$

The above numbers indicate that a hair color lightening composition of the invention damages hair much less than a conventional hair color lightening product.

Color Change/Color Control:

A conventional bleaching system gives the color change delta E of 10 to 65 on brown hair, with one treatment. In conventional hair bleaching treatments, consumers have little or no control of color, control of hue and control of lightening.

With the compositions and methods of the invention, there can be can delivered delta E of 0.1 to 65 on brown hair. Also, because hair lightening by the methods of the present invention is done in gradual, stepwise manner, the consumer has a great deal of control over the hue and lightening of her hair.

With compositions of the present invention there is no appreciable hair damage no matter how many application treatments according to the invention are performed.

At the same time that hair damage is avoided by the methods of the invention, the consumer's hair is gradually being brought to the desired shade and color. This gradual change of color lightening has two advantages: first, since the color lightening is changed gradually, the consumer can stop the process if she does not like the color shade her hair is turning to. Second, some consumers do not want an abrupt change in color lightening because they may feel embarrassed in public after having made such an abrupt change to the color shade of their hair. Moreover, the method and compositions of the present invention can be used in the shower, and on a frequent or daily basis, because the compositions and methods of the present invention, by contrast with conventional color lightening compositions, do not employ poisonous levels of chemicals and also because compositions and methods of the present invention involve hair application steps that can be up to about two minutes in length. By contrast conventional hair color lightening compositions require approximately 30 minutes' time for each application, an amount of time, which is clearly not suitable for use in the shower.

The methods of the present invention are not as messy as conventional hair color lightening methods. The methods of the present invention do not use chemical compositions that are as smelly and noxious as those used in conventional permanent hair-color lightening methods. Because the methods of the invention can be carried out in the shower, they do not involve the dripping and the mess associated with conventional hair color lightening, which are often done over the bathroom sink.

A dual package which can be employed in the products and kits of the present invention is disclosed in U.S. Pat. No. 6,082,588 to Markey et al which is hereby incorporated by reference.

Kit Containing an Instruction Sheet

The present invention also relates to a kit for carrying out the hair color lightening method of the invention. The kit comprises an aqueous ammonia gel base or conditioner base (part ai) and an oxidizing compound (part aii) each in a separate container or in a dual container, as described herein. The kit also contains written instructions that explain how the compositions of the invention are used.

The consumer admixes the components of the kit according to written instructions, to obtain the aqueous reaction mixture. The mixing may be conducted in a separate vessel external to the kit, or may take place in a container of the kit adapted to provide sufficient head space for mixing. The components that are mixed are the part ai and part aii. Part ai and part aii may also be admixed on the hair of the user. Essentially upon mixing, the reaction will commence. After treatment for a desired time the mixture may be removed, preferably with water or a conventional shampoo or a conventional conditioning shampoo.

As noted above, peroxygen composition and gel composition are mixed together, and the resulting mixture may be applied to the hair and allowed to remain for a set time, usually about 1 to about 2 minutes.

Desired change in hair color lightening by the methods of the invention is set forth by the mathematical formulas described above. Desired change in hair color lightening can be achieved in a number of other ways. In the first instance, the consumer can compare her hair color lightening with desired hair color or the hair color of a sample tress. Hair lightening by the gradual, stepwise method of the invention can be repeated until her hair color matches the desired hair color. It is noted that the compositions used in the gradual stepwise methods of the invention have lower contact times and thus repeated use of these compositions will not cause hair damage as do conventional hair color lightening products. An unexpected discovery of the present invention is that for damage to hair to occur, contact time in each treatment must go above the threshold value of about two minutes. Thus, the consumer can lighten the color of the hair through repeated applications wherein the duration of each color lightening application can be about two minutes or less. The following two advantages are thereby achieved: first, there is a stepwise approach to the desired color shade; and second, a minimum of hair damage is done.

By the methods of the invention, durable desired hair color shade, with a minimum of hair damage is achieved.

In the gradual stepwise method of the invention, the consumer can also compare the color of her hair with the desired hair color shade, which can be printed on the package of the product. The consumer can also vary the number of days of application of the product, and the consumer can also vary the amount of time the mixture of part ai and part aii is left in the hair on each application. The number of applications can vary from about 7 to about 30 applications. The time of each application can vary from about 1 to about two minutes. Alternatively, the consumer can use the compositions of the invention for an indefinite number of steps in order to maintain hair color at a certain desired shade.

The gradual, stepwise method of the invention occurs over the course of days. Therefore, the final color shade of the consumer's hair may be affected by the amount of exposure to the sun of the hair during the course of treatment.

Desired hair color lightening can also be reached by using the gradual stepwise method of the invention and comparing the hair after each treatment until it matches hair tresses taken from the consumer during a prior treatment.

Desired hair color lightening can also be reached by using the gradual stepwise method of the invention and testing the hair after each treatment with instruments, which measure the color shade of the hair. When the measurements of hair color lightening of the treated hair reach a desired level, the treatment can be stopped.

Indeed, reaching the desired hair color shade can be achieved by the use of any matching or comparison method commonly employed in the art.

What is claimed is:

1. A method for lightening the color of hair which comprises subjecting said hair to a number of treatments, having a set time interval between each two consecutive such treatments, wherein each treatment comprises steps a.) and b.) below:
a.) contacting said hair, for a period of about 5 seconds to about 5 minutes with a recently made mixture of:
Part ai: ammonia in gel base or conditioner base at alkaline pH;
Part aii: oxidizing compound in water with conditioning ingredients at acidic pH;
b.) rinsing said mixture from said hair with water;
and wherein said number of treatments is between about 2 to about 30; and wherein said set time interval between each two consecutive treatments is between about 8 hours and 30 days.

2. A method according to claim 1 wherein said oxidizing compound in part aii is present at about 2% to about 15%.

3. A method according to claim 1 wherein said ammonia in part ai is present at about 2% to about 10%.

4. A method according to claim 1, wherein said part ai prior to mixture with part aii has a pH of about 8 to about 12.

5. A method according to claim 1, wherein part aii prior to mixture with part ai has a pH of about 3 to about 5.

6. A method according to claim 1 wherein said period for contacting said hair is between about 1 minute and 3 minutes.

7. A method according to claim 1 wherein said set time interval between each two consecutive treatments is between about 1 day and about 3 days.

8. A method according to claim 1 wherein said hair has combing index in the range of 1.1 to 4.0.

9. A method according to claim 1 wherein said hair has combing force in the range of 5 to 55 gm force.

10. A method according to claim 1 wherein said hair has break stress in the range of 0.005 to 0.03 gm force/micron.

11. A method according to claim 1 wherein said composition delivers delta E of 0.1 to 65 on brown hair and delta E of 0.1 to 15 on blonde hair.

12. A method according to claim 1 wherein the ratio IR absorption at 1040 cm−1/1240 cm−1 is in the range of 0.01 to 1.5.

13. A method according to claim 1 wherein said oxidizing compound is selected from the group consisting of hydrogen peroxide, urea peroxide, melamine peroxide, sodium perborate and sodium percarbonate.

14. A method according to claim 1 wherein the part ai comprises from about 35% to about 98.9% water.

15. A method according to claim 1, wherein the mixture of part ai and part aii has a neat viscosity of from about 500 cps to about 60,000 cps at 26.7 degrees C., as measured by a Brookfield RVTDCP with a spindle CP-41 at 1 RPM for 3 minutes.

16. A method for maintaining hair color shade through the use of a hair color lightening composition which comprises subjecting said hair to successive treatments, having a set time interval between each two consecutive such treatments, wherein each treatment comprises steps a.) and b.) below:
a.) contacting said hair, for a period of about 5 seconds to about 5 minutes with a recently made mixture of:
Part ai: ammonia gel at alkaline pH;
Part aii: oxidative compound in water with conditioning ingredients at acidic pH;
b.) rinsing said mixture from said hair with water;
and wherein said set time interval between each two consecutive treatments is between about 8 hours and 30 days.

17. A dispenser for dispensing simultaneously or nearly simultaneously part ai and part aii according to claim 1, which comprises:
A.) a means for holding part ai and part aii in physically separate locations;
B.) a means for protecting part ai and part aii from air prior to dispensing;
C.) a means for dispensing part ai and part aii in approximately equal amounts and in physical proximity to each other.

18. A method according to claim 1 wherein part ai and part aii are mixed by hand.

19. A method according to claim 1 carried out in a shower.

20. A method according to claim 1 which comprises rinsing said mixture of part ai and part aii from said hair with water in a shower.

21. A composition for lightening hair which comprises a mixture of
part ai of about 0.1 to about 99.9% of an aqueous ammonia gel base or conditioner base at alkaline pH; and
part aii an oxidizing compound in water with conditioning agents at acidic pH.

22. A composition in accordance with claim 19 wherein part ai further comprises a volatile silicone.

23. A composition in accordance with claim 19 which further comprises a long chain fatty alcohol selected from the group consisting of stearyl alcohol, ceteareth-20, cetyl alcohol and mixtures thereof.
